(19) United States
(12) Patent Application Publication
(10) Pub. No.: US 2015/0148794 A1
(43) Pub. Date: May 28, 2015

(54) LOW PROFILE MEDICAL DEVICES FOR SYMPATHETIC NERVE ABLATION

(71) Applicant: BOSTON SCIENTIFIC SCIMED, INC., Maple Grove, MN (US)

(72) Inventors: ROBERT N. SQUIRE, MAPLE GROVE, MN (US); JEFFREY S. LINDQUIST, MAPLE GROVE, MN (US); DEREK C. SUTERMEISTER, HAM LAKE, MN (US); PATRICK A. HAVERKOST, BROOKLYN CENTER, MN (US); TIMOTHY A. OSTROOT, COKATO, MN (US)

(73) Assignee: BOSTON SCIENTIFIC SCIMED, INC., MAPLE GROVE, MN (US)

(21) Appl. No.: 14/549,121

(22) Filed: Nov. 20, 2014

Related U.S. Application Data
(60) Provisional application No. 61/908,517, filed on Nov. 25, 2013.

(51) Int. Cl. A61B 18/14

(52) U.S. Cl. CPC A61B 18/1492; A61B 2018/00511
USPC 606/41; 29/25.01

(57) ABSTRACT

Medical devices and methods for making and using medical devices are disclosed. An example medical device for sympathetic nerve ablation may include a catheter shaft. An expandable balloon may be coupled to the catheter shaft. The balloon may be capable of shifting between an unexpanded configuration and an expanded configuration. The balloon may include a first layer and a second layer. The first layer may include a convertible circuit. An electrode may be coupled to the balloon and may be in electrical contact with the convertible circuit.

### CROSS-REFERENCE TO RELATED APPLICATIONS

This application claims priority under 35 U.S.C. §119 to U.S. Provisional Application Ser. No. 61/908,517, filed Nov. 25, 2013, the entirety of which is incorporated herein by reference.

### TECHNICAL FIELD

The present disclosure pertains to medical devices, and methods for manufacturing medical devices. More particularly, the present disclosure pertains to medical devices for sympathetic nerve ablation.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device for sympathetic nerve ablation may include a catheter shaft. An expandable balloon may be coupled to the catheter shaft. The balloon may be capable of shifting between an unexpanded configuration and an expanded configuration. The balloon may include a first layer and a second layer. The first layer may include a convertible circuit. An electrode may be coupled to the balloon and may be in electrical contact with the convertible circuit.

An example method for manufacturing a medical device may include providing a tubular member including a first layer and a second layer. The first layer may include a polymer loaded with organic semi-conductive metallic particles. The method may also include laser heat-activating the first layer to define a conductive trace in the first layer and coupling an electrode to the conductive trace.

An example method for manufacturing a medical device may include providing a tubular member including a first layer and applying a coating to the first layer. The coating may include organic semi-conductive metallic particles. The method may also include laser heat-activating at least a portion of the coating to define a conductive trace in the coating and coupling an electrode to the conductive trace.

Another example medical device for sympathetic nerve ablation may include a catheter shaft. An expandable balloon may be attached to the catheter shaft. The balloon may include a first layer and a second layer. The first layer may include a laser heat-activated section defining a conductive region in the first layer. An electrode may be coupled to the balloon and may be in electrical contact with the conductive region.

Another example medical device for sympathetic nerve ablation may include a catheter shaft. An expandable balloon may be coupled to the catheter shaft. The balloon may be capable of shifting between an unexpanded configuration and an expanded configuration. The balloon may include a first layer and a second layer. The first layer may include a circuit capable of shifting between a non-conductive state and a conductive state. An electrode may be coupled to the balloon and may be in electrical contact with the circuit.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an example sympathetic nerve ablation system;

FIG. 2 is a perspective view of an example expandable member of a sympathetic nerve ablation device;

FIG. 3 is a partial cross-sectional side view of an example tubular member;

FIG. 4 is a cross-sectional side view of a portion of an example medical device;

FIG. 5 is a cross-sectional side view of a portion of another example medical device;

FIG. 6 is a cross-sectional side view of an example tubular member;

FIG. 7 is a cross-sectional view taken through line 7-7 in FIG. 6;

FIGS. 8-13 illustration some aspects of an example method for manufacturing a medical device;

FIG. 14 is a cross-sectional view of a portion of an example medical device;

FIG. 15 is a cross-sectional view of a portion of an example medical device; and

FIG. 16 is a side view of a portion of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangable with each other to form other additional embodiments or to complement and/or enrich the described embodiment(s), as would be understood by one of ordinary skill in the art.

Certain treatments are aimed at the temporary or permanent interruption or modification of select nerve function. In some embodiments, the nerves may be sympathetic nerves. One example treatment is renal nerve ablation, which is sometimes used to treat conditions such as or related to hypertension, congestive heart failure, diabetes, or other conditions impacted by high blood pressure or salt retention. The kidneys produce a sympathetic response, which may increase the undesired retention of water and/or sodium. The result of the sympathetic response, for example, may be an increase in blood pressure. Ablating some of the nerves running to the kidneys (e.g., disposed adjacent to or otherwise along the renal arteries) may reduce or eliminate this sympathetic response, which may provide a corresponding reduction in the associated undesired symptoms (e.g., a reduction in blood pressure).

Some embodiments of the present disclosure relate to a power generating and control apparatus, often for the treatment of targeted tissue in order to achieve a therapeutic effect. In some embodiments, the target tissue is tissue containing or proximate to nerves. In other embodiments, the target tissue(s) are sympathetic nerves, including, for example, sympathetic nerves disposed adjacent to blood vessels. In still other embodiments the target tissue is luminal tissue, which may further comprise diseased tissue such as that found in arterial disease.

In some embodiments of the present disclosure, the ability to deliver energy in a targeted dosage may be used for nerve tissue in order to achieve beneficial biologic responses. For example, chronic pain, urologic dysfunction, hypertension, and a wide variety of other persistent conditions are known to be affected through the operation of nervous tissue. For example, it is known that chronic hypertension that may not be responsive to medication may be improved or eliminated by disabling excessive nerve activity proximate to the renal arteries. It is also known that nervous tissue does not naturally possess regenerative characteristics. Therefore it may be possible to beneficially affect excessive nerve activity by disrupting the conductive pathway of the nervous tissue. When disrupting nerve conductive pathways, it is particularly advantageous to avoid damage to neighboring nerves or organ tissue. The ability to direct and control energy dosage is well-suited to the treatment of nerve tissue. Whether in a heating or ablating energy dosage, the precise control of energy delivery as described and disclosed herein may be directed to the nerve tissue. Moreover, directed application of energy may suffice to target a nerve without the need to be in exact contact, as would be required when using a typical ablation probe. For example, eccentric heating may be applied at a temperature high enough to denature nerve tissue without causing ablation and without requiring the piercing of luminal tissue. However, it may also be desirable to configure the energy delivery surface of the present disclosure to pierce tissue and deliver ablating energy similar to an ablation probe with the exact energy dosage being controlled by a power control and generation apparatus.

In some embodiments, efficacy of the denervation treatment can be assessed by measurement before, during, and/or after the treatment to tailor one or more parameters of the treatment to the particular patient or to identify the need for additional treatments. For instance, a denervation system may include functionality for assessing whether a treatment has caused or is causing a reduction in neural activity in a target or proximate tissue, which may provide feedback for adjusting parameters of the treatment or indicate the necessity for additional treatments.

Many of the devices and methods described herein are discussed relative to renal nerve ablation and/or modulation. However, it is contemplated that the devices and methods may be used in other treatment locations and/or applications where sympathetic nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, cardiac ablation, pain management, pulmonary vein isolation, pulmonary vein ablation, tumor ablation, benign prostatic hyperplasia therapy, nerve excitation or blocking or ablation, modulation of muscle activity, hyperthermia or other warming of tissues, etc. The disclosed methods and apparatus can be applied to any relevant medical procedure, involving both human and non-human subjects. The term modulation refers to ablation and other techniques that may alter the function of affected nerves and other tissue.

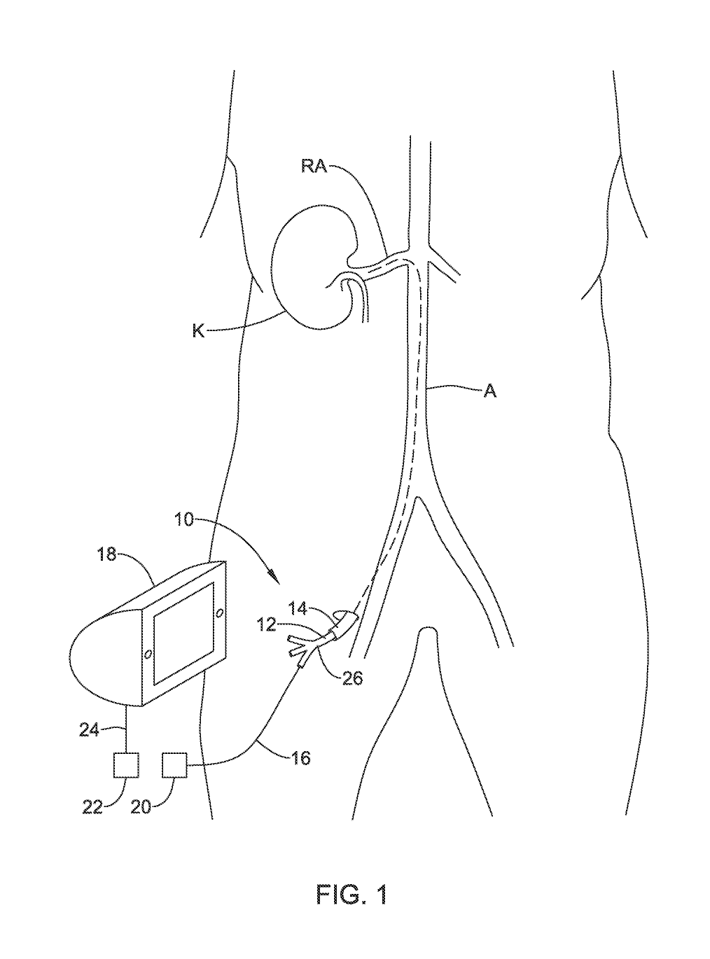

FIG. 1 is a schematic view of an example sympathetic nerve ablation system 10. System 10 may include a sympathetic nerve ablation device 12. Sympathetic nerve ablation device 12 may be used to ablate nerves (e.g., renal nerves) disposed adjacent to the kidney K (e.g., renal nerves disposed about a renal artery RA). In use, sympathetic nerve ablation device 12 may be advanced through a blood vessel such as the aorta A to a position within the renal artery RA. This may include advancing sympathetic nerve ablation device 12 through a guide sheath or catheter 14. When positioned as desired, sympathetic nerve ablation device 12 may be activated to activate one or more electrodes (not shown). This may include operatively coupling sympathetic nerve ablation device 12 to a control unit 18, which may include an RF generator, so as to supply the desired activation energy to the electrodes. For example, sympathetic nerve ablation device 12 may include a wire or conductive member 16 with a first connector 20 that can be connected to a second connector 22 on the control unit 18 and/or a wire 24 coupled to the control unit 18. In at least some embodiments, the control unit 18 may also be utilized to supply/receive the appropriate electrical energy and/or signal to activate one or more sensors disposed at or near a distal end of sympathetic nerve ablation device 12. When suitably activated, the one or more electrodes may be capable of ablating tissue (e.g., sympathetic nerves) as described below and the one or more sensors may be used to detect desired physical and/or biological parameters.

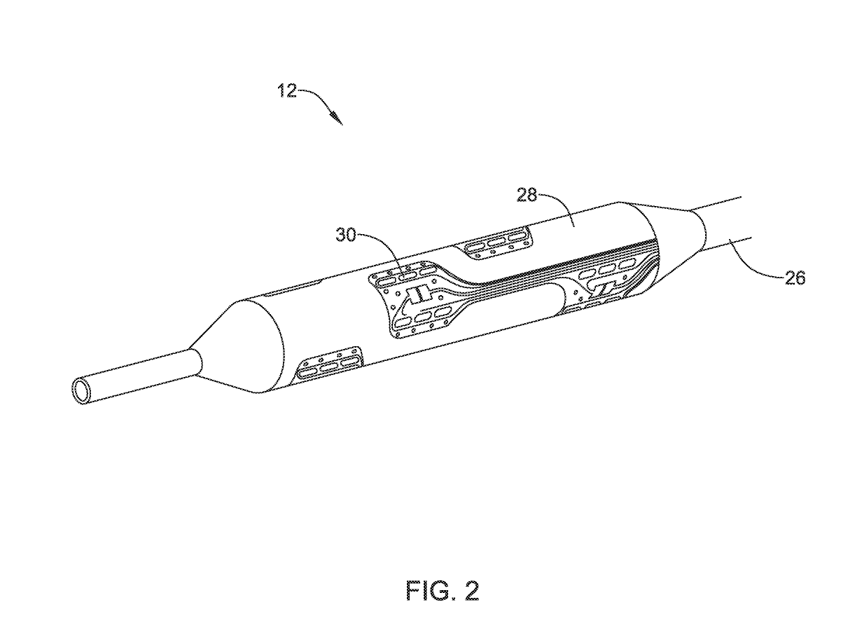

In some embodiments, the sympathetic nerve ablation device 12 may include an elongate tubular member or catheter shaft 26, as shown in FIG. 2. In some embodiments, the elongate tubular member or catheter shaft 26 may be configured to be slidingly advanced over a guidewire or other elongate medical device to a target site. In some embodiments, the elongate tubular member or catheter shaft 26 may be configured to be slidingly advanced within a guide sheath or catheter 14 to a target site. In some embodiments, the elongate tubular member or catheter shaft 26 may be configured to be advanced to a target site over a guidewire, within a guide sheath or catheter 14, or a combination thereof. An expandable member 28 may be disposed at, on, about, or near a distal region of the elongate tubular member or catheter shaft 26. In some embodiments, the expandable member 28 may be a compliant or a non-compliant balloon. In some embodiments, the expandable member 28 may be capable of shifting between an unexpanded configuration and an expanded configuration. In some embodiments, one or more electrode assemblies 30 may be arranged on the expandable member 28. Electrode assemblies 30 may resemble those disclosed in U.S. patent application Ser. No. 13/750,879, the entire disclosure of which in herein incorporated by reference. For example, electrode assemblies 30 may include a flexible substrate or circuit having one or more electrodes coupled thereto.

A number of ablation medical devices utilize electrodes that are adhered to the exterior surface of an inflatable balloon. While these devices may be effective, the electrodes or electrode assemblies may add to the profile of the device. Furthermore, when used with a balloon, the added material on the exterior of the balloon could inadvertently be cleaved from or otherwise delaminated from the balloon when attempting to draw the device back into a guide catheter or sheath. The devices disclosed herein may include electrodes that are incorporated into a medical device in a way that has little or no impact on the profile of the device. Accordingly, the devices disclosed herein may be considered “low profile” medical devices and may include electrodes that may be less likely to catch or otherwise separate from the device during use and/or repositioning.

The devices disclosed herein may utilize a tubular member as a starting material. At least a portion or layer of the tubular member may include a polymer loaded with organic semi-conductive metallic particles. The particles may be incorporated into or otherwise dispersed throughout the tubular member, through a portion of the tubular member, through a region of the tubular member, through a portion of or all of a layer of the tubular member, or the like. The particles can be activated so as to become conductive. Accordingly, conductive layers or conductive traces (to which an electrode may be electrically coupled to) may be defined in the tubular member itself. This differs from other devices where a conductive layer is attached to a shaft or device. Because the conductive layer is a portion within the tubular member, the profile of the tubular member (and the medical device incorporating the tubular member) can be kept to a minimum. Furthermore, because the tubular member may be formed from a polymer, the three dimensional structure of the tubular member can be manufactured to have essentially any suitable shape including relatively complex three-dimensional shapes. This may allow for greater variety in the shapes possible for forming ablation medical devices.

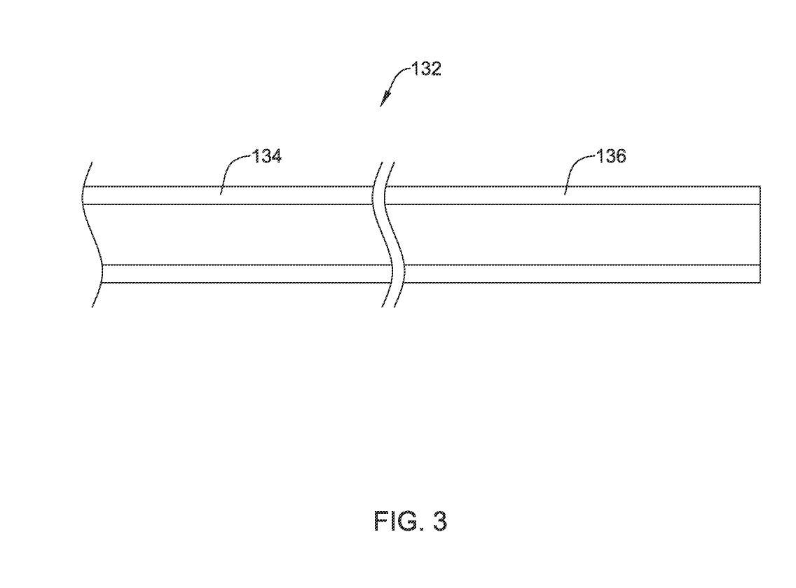

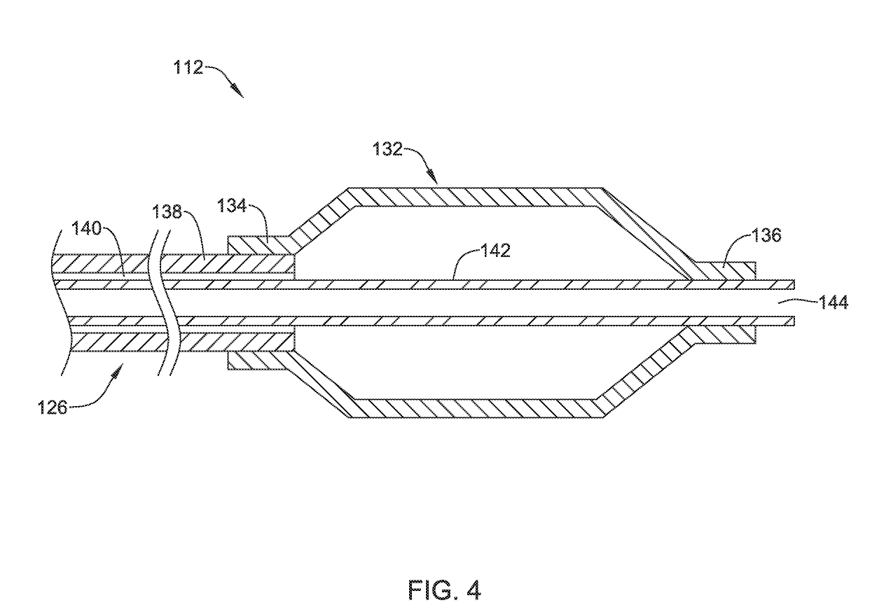

The tubular members, as referred to herein, may be utilized to form a portion of or substantially all of a medical device (e.g., an ablation device). For example, FIG. 3 schematically illustrates an example tubular member 132 having a proximal portion 134 and a distal portion 136. FIG. 3 is meant to be schematic in nature and is not intended to show every feature of tubular member 132. Indeed, tubular member 132 may be similar in form and function to other tubular members disclosed herein. Proximal portion 134 may be the proximal end of a medical device or may simply be the proximal portion of a portion or component of a medical device. For example, FIG. 4 illustrates an example medical device 112. Medical device 112 may include catheter shaft 126. Catheter shaft 126 may include an outer tubular member 138 and an inner tubular member 142. Inner tubular member 142 may define a guidewire lumen 144. An inflation lumen 140 may be defined between outer tubular member 138 and inner tubular member 142.

In the example shown in FIG. 4, tubular member 132 includes or otherwise takes the form of a balloon. Balloon 132 may be attached to catheter shaft 126. Accordingly, proximal portion 134 may be a proximal waist of balloon 132 and may be attached to outer tubular member 138. Distal portion 136 may be a distal waist of balloon 132 and may be attached to inner tubular member 142. This is just an example. Other forms and/or configurations are contemplated.

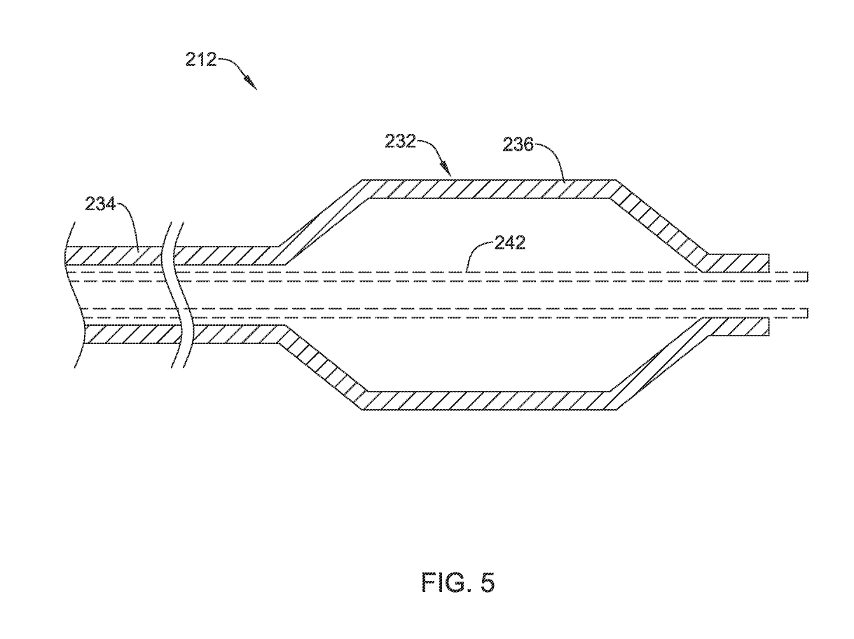

FIG. 5 illustrates another example medical device 212 utilizing tubular member 232. In this example, tubular member 232 is a unitary structure where distal portion 236 is a balloon or balloon portion and proximal 234 is a shaft or shaft portion. In some embodiments, medical device 212 may also include inner tubular member 242.

Collectively, FIGS. 4-5 illustrate some of the configurations contemplated for the tubular members disclosed herein that ultimately may make up a portion of or substantially all of a medical device (e.g., a nerve ablation and/or modulation medical device). The following figures (and corresponding description) outline some of the details regarding the structure and construction of tubular members and medical devices utilizing tubular members.

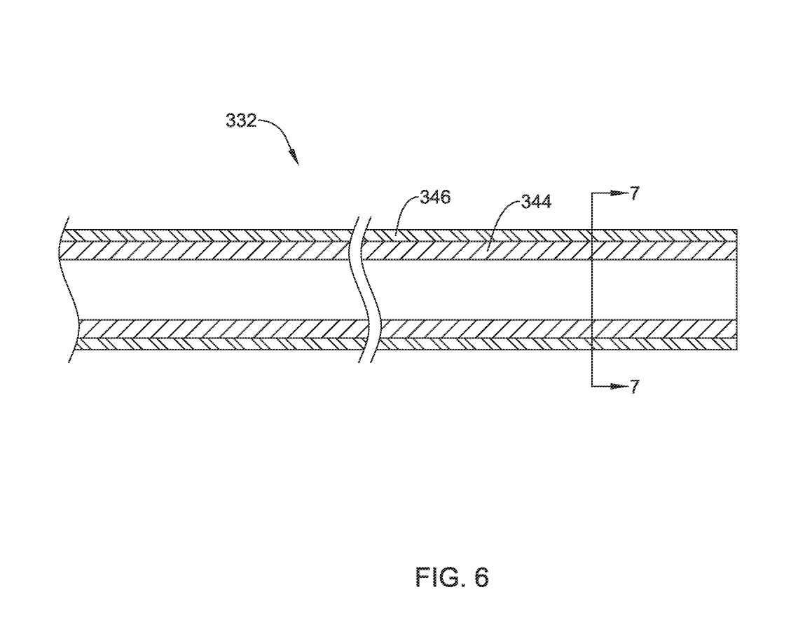

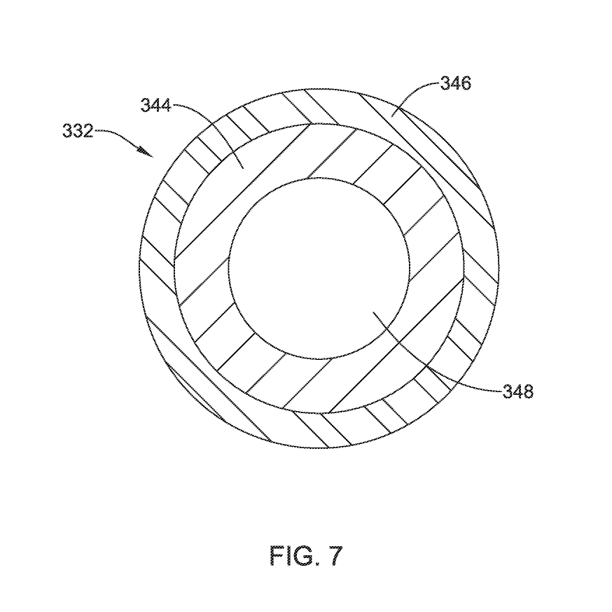

FIGS. 6-7 illustrates tubular member 332. Tubular member 332 may be used to manufacture at least a portion of a medical device such as a nerve ablation and/or modulation device. Tubular member 332 may include a first or inner layer 344 and a second or outer layer 346. A lumen 348 may be defined within tubular member 332. In some embodiments, layer 344 may account for about 25-95% of the total thickness of tubular member 332, or about 30-90% of the total thickness of tubular member 332, or about 70-90% of the total thickness of tubular member 332. Layer 344 may include a polymeric material such as a polyether block amide, a polyethylene terephthalate, or a nylon. Other materials are contemplated including those materials disclosed herein.

In some embodiments, layer 346 may account for about 5-75% of the total thickness of tubular member 332, or about 10-70% of the total thickness of tubular member 332, or about 10-30% of the total thickness of tubular member 332. Layer 346 may be disposed on layer 344 via a co-extrusion process, spray coating, brush coating, dip coating, or the like, or any other suitable process.

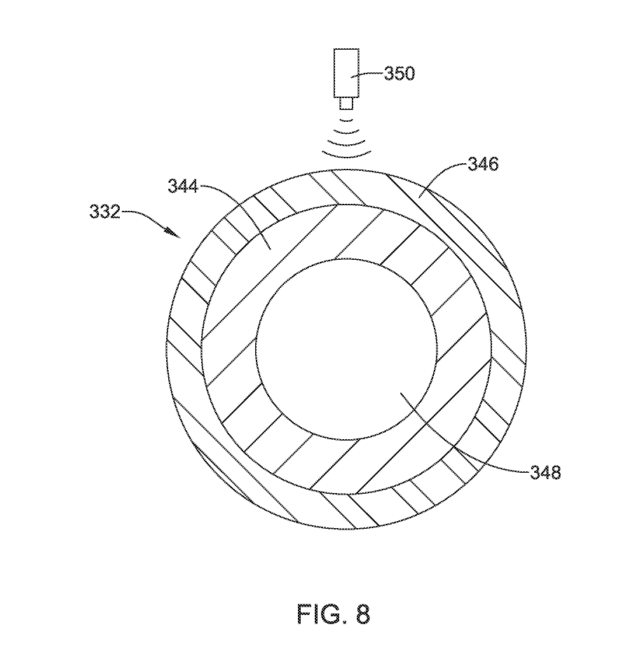

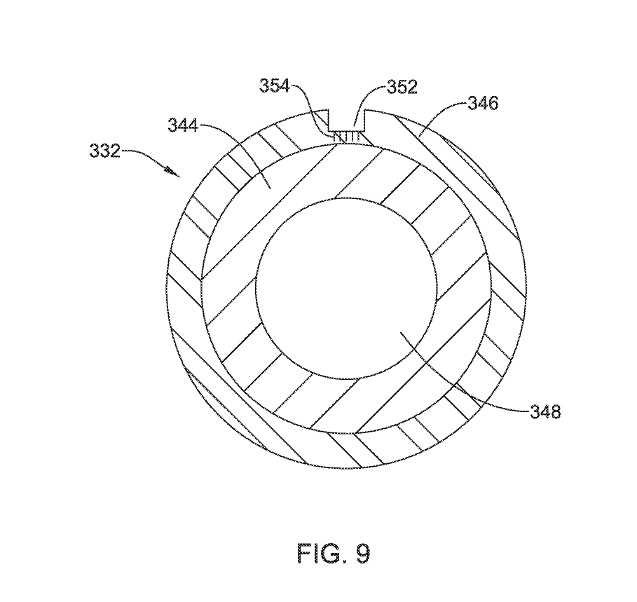

Layer 346 may include a polymer doped with or otherwise including organic semi-conductive metallic particles. In at least some embodiments, the organic semi-conductive metallic particles may be dispersed throughout layer 346. However, in other embodiments, the organic semi-conductive metallic particles may be disposed within a portion of layer 346, applied to a surface or region of organic semi-conductive metallic particles, or the like. Initially, the organic semi-conductive metallic particles may be in a “non-activated” or non-conductive state. The organic semi-conductive metallic particles may be activated or otherwise converted to an “activated” or conductive state by a laser assembly 350 as shown in FIG. 8. Laser assembly 350 may heat, sinter, and/or otherwise activate the organic semi-conductive metallic particles so as to define a conductive region 354 as shown in FIG. 9. For example, when energy from the laser beam makes contact with the organic metallic particles, the laser energy may convert the metallic doping material into conductive traces via sintering/metallization process along the areas of layer 346 exposed to laser energy. This creates the “conductive trace” for electro-plating circuitry metal material buildup or otherwise the deposition of electrodes onto the “conductive trace”.

The laser activation process, in at least some embodiments, may also etch or remove a portion of layer 346 to define an etched region 352 in layer 346. The laser activation process can be controlled so as to tailor the depth of etched region 352 or otherwise define the desired shape or pattern in tubular member 332. In some embodiments, three-dimensional modeling (e.g., using a suitable modeling software such as AUTOCAD, SOLIDWORKS, PRO-ENGINEER, or the like) may be utilized to form the desired structure or form in tubular member 332 so as to ultimately define a number of different electrode assembly shapes. This may include a variety of different shapes for the ultimate electrode assemblies including relatively complex shapes.

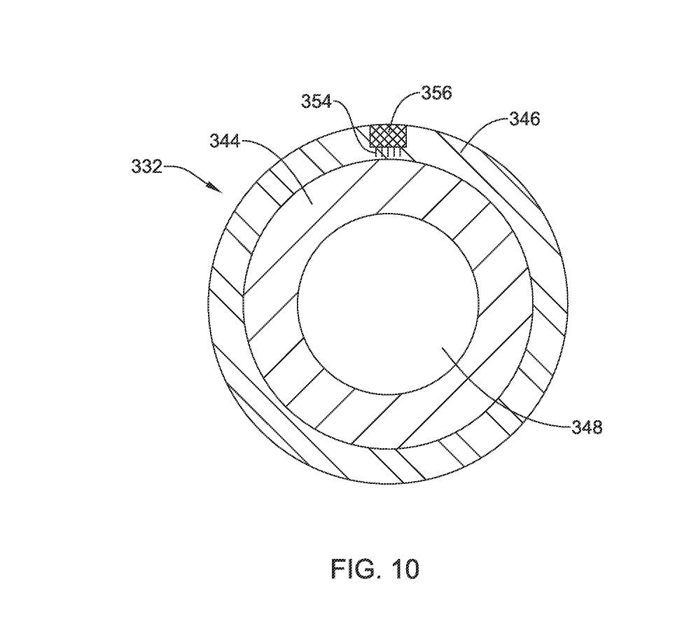

An electrode 356 may be deposited within or along conductive region 354 as shown in FIG. 10. This may include depositing a suitable material adjacent to conductive region 354 and/or etched region 352. In some embodiments, electrode 356 may be a discrete electrode. In other embodiments, sets or patterns of electrodes 356 may be deposited along tubular member 332. Electrode 356 may be formed from any suitable conductive material such as copper, gold, or the like. Depositing electrode 356 may include an electroplating process, a deposition process, or another suitable process.

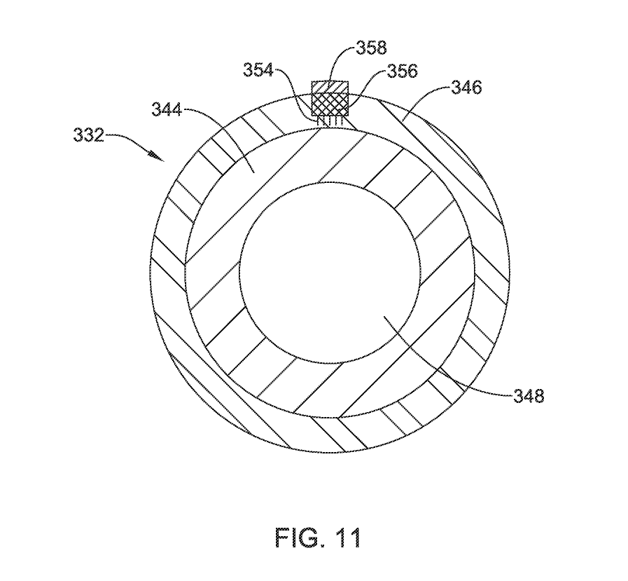

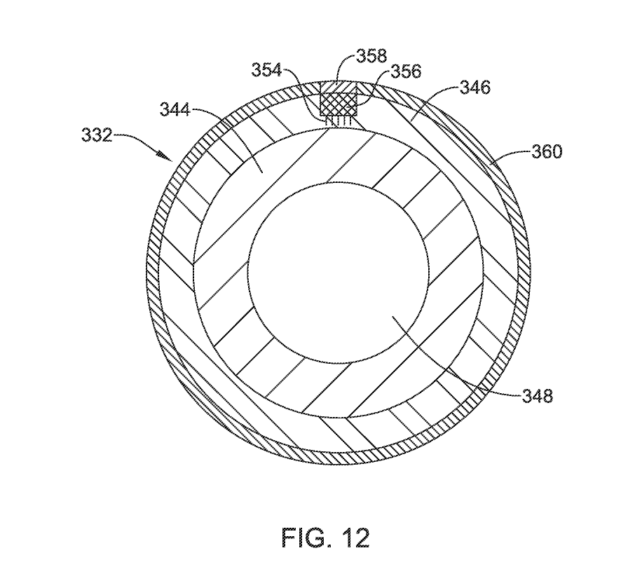

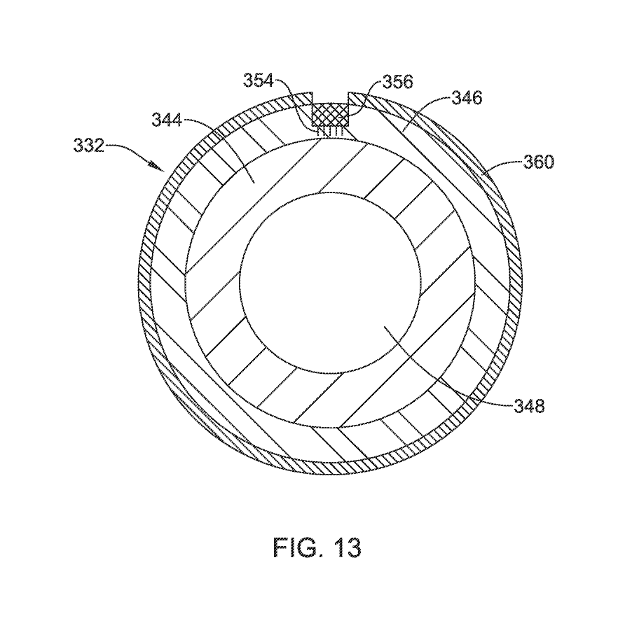

In some embodiments, it may be desirable to insulate a portion of conductive region 354. Insulating may include disposing a mask 358 over electrode 356 as shown in FIG. 11. An insulating layer 360 may be disposed along portions of tubular member 332 as shown in FIG. 12. Insulating layer 360 may include any suitable insulating material. For example, insulating layer 360 may include an aliphatic polyether-based thermoplastic polyurethanes such as TECOFLEX (e.g., TECOFLEX SG-60d), a poly(p-xylylene) polymer such as PARYLENE C, or the like. Other materials are contemplated. In some embodiments, following the application of insulating layer 360, mask 358 may be removed as shown in FIG. 13.

In the embodiment shown in FIG. 13, electrode 356 may be disposed radially inward relative to the outermost layer of tubular member 332 (e.g., insulating layer 360). This may help to reduce the likelihood of electrode 356 “catching” if tubular member 332 was to be retracted into a device (e.g., within a guide catheter or sheath).

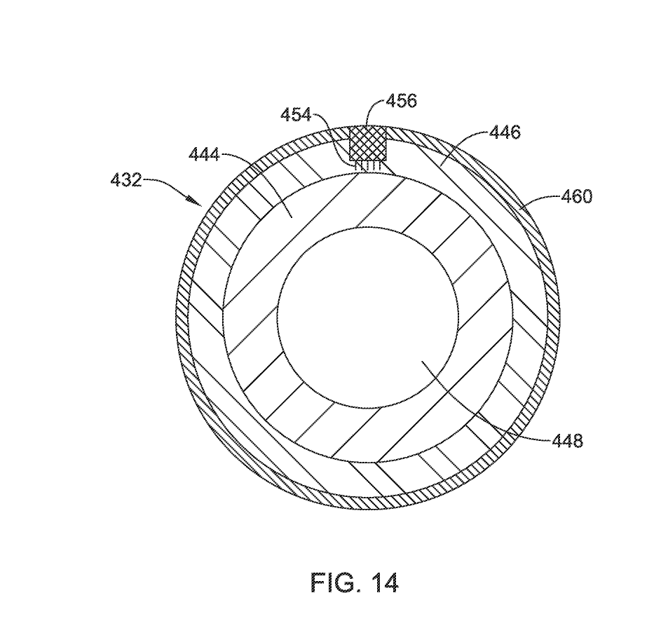

FIG. 14 illustrates tubular member 432 including layer 444, layer 446, and lumen 448. Layer 446 may include conductive region 454. Electrode 456 may be coupled to conductive region 454. Insulating layer 460 may be disposed over at least a portion of conductive region 454. According to this embodiment, electrode 456 may be radially aligned with the outermost layer of tubular member 432 (e.g., insulating layer 460).

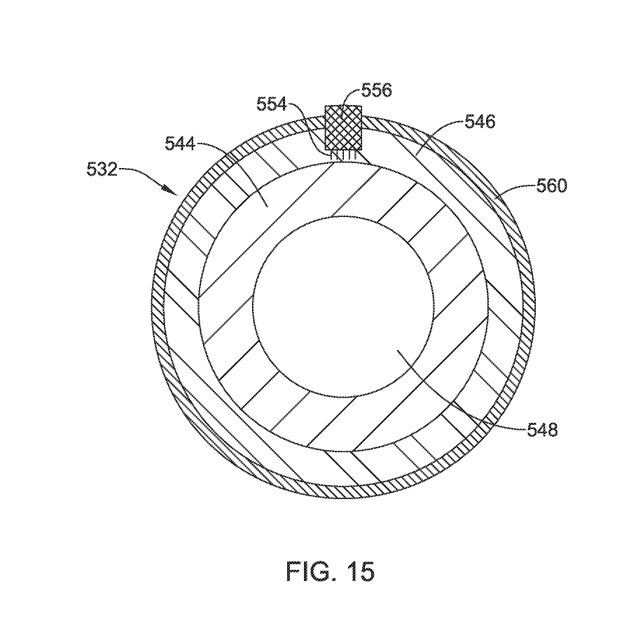

FIG. 15 illustrates tubular member 452 including layer 544, layer 546, and lumen 548. Layer 546 may include conductive region 554. Electrode 556 may be coupled to conductive region 554. Insulating layer 560 may be disposed over at least a portion of conductive region 554. According to this embodiment, electrode 556 may be disposed radially inward relative to the outermost layer of tubular member 532 (e.g., insulating layer 560).

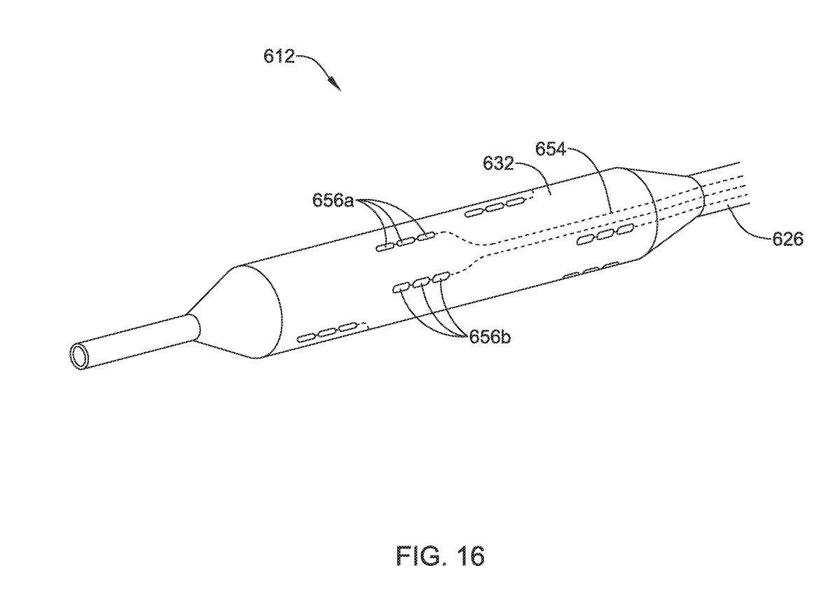

The process disclosed herein may be used to define conductive region 354/454/545 and one or more electrodes 356/456/556 in tubular member 332/432/532. Part of this process may also include forming tubular member 332 into a balloon (e.g., using a blow-molding process or the like), into a catheter shaft or region of a catheter shaft, into both a balloon and a catheter shaft (or catheter shaft region), or the like. FIG. 16 illustrates another example medical device 612 incorporating some of the aspects of disclosure. Device 612 may include shaft 626 and balloon 632. A plurality of electrodes including electrodes 656a and electrodes 656b may be disposed along balloon 632. Electrodes 656a/656b may be coupled to conductive region(s) 654. Because conductive region(s) 654 are formed within the material of balloon 632, electrodes 656a/656b (and the corresponding conductive regions 654 coupled thereto) may have little or no impact on the profile of device 612. For example, electrodes 656a/656b may be disposed at or radially inward relative to the outer surface of balloon 632 and conductive regions 654 may be disposed “within” balloon 632 (and also may be disposed within shaft 626).

The form of device 612 may vary. For example, the number, arrangement, and configuration of electrodes 656a/656b may vary. In at least some embodiments, electrodes 656a/656b may define pairs of bipolar electrodes. In some of these and in other embodiments, electrodes 656a/656b may include one or more monopolar electrodes.

The materials that can be used for the various components of the ablation device 12 (and/or other devices disclosed herein) may include those commonly associated with medical devices. For simplicity purposes, the following discussion makes reference to the ablation device 12. However, this is not intended to limit the devices and methods described herein, as the discussion may be applied to other similar tubular members and/or expandable members and/or components of tubular members and/or expandable members disclosed herein.

The ablation device 12 and the various components thereof may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride

(PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or on-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions of the ablation device 12 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the ablation device 12 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the ablation device 12 to achieve the same result.

In some embodiments, a degree of Magnetic Resonance Imaging (MRI) compatibility may be imparted into the ablation device 12. For example, portions of device, may be made of a material that does not substantially distort the image and create substantial artifacts (i.e., gaps in the image). Certain ferromagnetic materials, for example, may not be suitable because they may create artifacts in an MRI image. In some of these and in other embodiments, portions of the ablation device 12 may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

U.S. patent application Ser. No. 13/750,879, filed on Jan. 25, 2013, entitled “METHODS AND APPARATUSES FOR REMODELING TISSUE OF OR ADJACENT TO A BODY PASSAGE”, now U.S. Patent Publication US20130165926A1 is herein incorporated by reference.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device for sympathetic nerve ablation, comprising:
a catheter shaft;
an expandable balloon coupled to the catheter shaft, the balloon being capable of shifting between an unexpanded configuration and an expanded configuration;
wherein the balloon includes a first layer and a second layer;
wherein the first layer includes a convertible circuit; and
an electrode coupled to the balloon and in electrical contact with the convertible circuit.

2. The medical device of claim 1, wherein the first layer includes a polymer loaded with organic semi-conductive metallic particles.

3. The medical device of claim 2, wherein the organic semi-conductive metallic particles are capable of shifting between a non-conductive state and a conductive state.

4. The medical device of claim 3, wherein the convertible circuit is defined by a region of the first layer where the organic semi-conductive metallic particles are in the conductive state.

5. The medical device of claim 4, wherein the organic semi-conductive metallic particles are shifted to the conductive state by heating.

6. The medical device of claim 4, wherein the organic semi-conductive metallic particles are shifted to the conductive state by laser heating.

7. The medical device of claim 4, wherein the organic semi-conductive metallic particles are shifted to the conductive state by laser heat sintering.

8. The medical device of claim 1, wherein the first layer further comprises a second convertible circuit.

9. The medical device of claim 1, wherein the convertible circuit is defined along an etched region of the first layer.

10. The medical device of claim 1, wherein the electrode has an outer surface that is disposed radially inward from an outer surface of the balloon.

11. The medical device of claim 1, wherein the electrode has an outer surface that is substantially radially aligned with an outer surface of the balloon.

12. The medical device of claim 1, wherein the electrode has an outer surface that is disposed radially outward from an outer surface of the balloon.

13. A method for manufacturing a medical device, the method comprising:
providing a tubular member including a first layer and a second layer;
wherein the first layer includes a polymer loaded with organic semi-conductive metallic particles;
laser heat-activating the first layer to define a conductive trace in the first layer; and
coupling an electrode to the conductive trace.

14. The method of claim 13, wherein the tubular member includes a catheter shaft portion and a balloon portion.

15. The method of claim 14, wherein laser heat-activating the first layer to define a conductive trace in the first layer includes laser heat-activating the first layer along the catheter shaft portion, along the balloon portion, or along both portions.

16. The method of claim 13, further comprising masking a portion of the first layer and applying an insulative coating to the tubular member.

17. A method for manufacturing a medical device, the method comprising:
providing a tubular member including a first layer;
applying a coating to the first layer, the coating including organic semi-conductive metallic particles;
laser heat-activating at least a portion of the coating to define a conductive trace in the coating; and
coupling an electrode to the conductive trace.

18. The method of claim 17, wherein the tubular member includes a catheter shaft portion and a balloon portion.

19. The method of claim 18, wherein laser heat-activating the coating to define a conductive trace in the coating includes laser heat-activating the coating along the catheter shaft portion, along the balloon portion, or along both portions.

20. The method of claim 17, further comprising masking a section of the coating and applying an insulating member to the tubular member.
